(19) United States
(12) Patent Application Publication
(10) Pub. No.: US 2004/0120851 A1
(43) Pub. Date: Jun. 24, 2004

(54) Apparati and method for remediating biologically active particles

(76) Inventor: James Aamodt, Sisters, OR (US)

Correspondence Address: GREENBERG-TRAURIG, 1750 TYSONS BOULEVARD, 12TH FLOOR, MCLEAN, VA, 22102, US

(21) Appl. No.: 10/323,602

(22) Filed: Dec. 19, 2002

(51) Int. Cl. A61L 9/00; B01J 19/00; A62B 7/08

(52) U.S. Cl. 422/28; 422/1; 422/5; 422/34; 422/37; 422/40; 422/120; 422/255

(57) ABSTRACT

The present invention provides a mixture and method for remediating air dispersed biological particles. The mixture contains a carrier, an active biocidal ingredient, and a deliquescent which are mixed, heated and then grinded into biocidal particles approximately 1-50 microns in size. The biocidal particles are then dispersed into the air infected with biological particles using an aerosol generator and similar device. The biocidal particles may be charged to attract the biological particles and the carrier assists in attaching to the biological particles. Upon exposure to ambient humidity the deliquescent dissolves and allows the active biocidal ingredient to react creating a chemical byproduct which attacks and remediates the biological particles. The present invention may utilize two mixtures with two separate active biocidal ingredients which react with one another to create the desired chemical byproduct such as chlorine dioxide. The chemical byproduct may be hydrogen peroxide. The active biocidal ingredient may be biocidal immediately through use of botanical active biocidal ingredients such as Cinnamon Leaf or Lemongrass.

### FIELD OF THE INVENTION

The present invention relates to apparati and processes for the remediation of harmful biological agents for use in combating biological attacks or accidents.

### BACKGROUND OF THE INVENTION

The threat of weapons of mass destruction being used on civilian populations such as the release or use of biological weapons and/or the release of biological agents has become a national and international health and safety concern. Biological weapons use either a bacteria or virus, or in some cases toxins which stem from bacteria, in an attempt to injure or kill. Of particular concern are the release of feared biological agents such as anthrax, smallpox, botulin toxin, and ebola virus. Still further, the concern is not only the release of such biological weapons against civilian populations but also the release against military personnel. The forms of deployment for such biological weapons include contamination of water sources, food sources, and direct contamination of the air.

The small particle sizes of the biological agents (1-50 microns) make them difficult to detect and remediate when released as spores or other ultra-fine airborne particles. There is a need to bind the biological particulates to precipitate them from the air, and also to remediate the harmful effect of the biological agent.

There are mechanical, physical, and chemical methods whereby objects, surfaces, and individuals may be decontaminated. Mechanical methods include washing, scrubbing and conventional flushing with water to mechanically remove the biological and/or bacteriological contaminants. This normally occurs without rendering the contaminants harmless. Physical methods include exposure to heat and radiation, such as ultraviolet radiation, although the effectiveness of these processes generally varies with humidity. Chemical methods are perhaps the most effective at rendering ineffective bioagents and may take the form of a liquid, gas, or aerosol treatment.

Commercial disinfectants recommended for general decontamination include phenol, isopropyl alcohol, dilute ammonia and chlorine solutions, formaldehydes, glutaraldehydes (inhalation of the fumes of which is hazardous), and chlorohexidine. Substantial quantities of these disinfectants must be obtained and stored well in advance of their actual need and transported for use in either small or large scale decontaminations.

Additional methods for decontaminating biological agents in the air or on surfaces include discharging an electrostaticly charged mist of hydrogen peroxide. Because hydrogen peroxide particles in a mist attract and cling to particles of biological agents, the joined particles grow in size and can settle out of the air more rapidly (“precipitate”), making them less of a threat. Precipitated particles can then be treated using an ultra-violet source which when combined with the hydrogen peroxide creates ozone to destroy the biological agents. However, this process has several known problems and requires a two step process. A major problem or concern is that the hydrogen peroxide mist rapidly evaporates in open or outside air deployments. Therefore, the use of a hydrogen peroxide mist against unconfined biological agent deployments is impractical.

Therefore, what is needed is an easy to use and effective solution which not only attracts and attaches to particles of active biological agents to precipitate such particles out of the air but that can also remediate the biological particles while they are still airborne and is effective against unconfined biological agent deployments.

### SUMMARY OF THE INVENTION

The present invention relates to a chemical attractant and method of use for the remediation of contaminated air and surfaces during and after a biological weapons attack or accidental exposure to biological agents.

Another object of the present invention is the dispersal of an aerosol or dry attractant during or after a biological attack or accident, wherein the attractant is rendered antimicrobial by impregnation of chemical compounds that are active biocidal ingredient alone or in combination.

To achieve these and other advantages and in accordance with the purpose of the present invention, as embodied and broadly described, in one aspect of the present invention there is provided a biocidal mixture for remediating a plurality of biological particles comprising: at least one carrier, at least one active biocidal ingredient and at least one deliquescent; wherein the at least one carrier, the at least one active biocidal ingredient and the at least one deliquescent are mixed, heated and ground into a plurality of particles approximately 1-50 microns in size; wherein when the plurality of biocidal particles are dispersed in the air, the deliquescent reacts with ambient humidity to facilitate binding with biological particles and to start a chemical reaction within the biocidal particle creating a chemical byproduct which remediates the biological threat posed by the biological particles.

In a further aspect of the present invention there is provided a biocidal mixture which comprises first and second active biocidal ingredients and contains at least one carrier and one deliquescent which are mixed, heated and ground into a plurality of active biocidal particles approximately 1-50 microns in size. The active biocidal particles are dispersed in the air with and the deliquescent reacts with ambient humidity to start a chemical reaction within the biocidal particle. The first and second active biocidal ingredient in the biocidal particles then react to create a chemical byproduct which remediates the biological particles.

In a further aspect of the present invention there is provided a first biocidal mixture comprised of a first active biocidal ingredient, a first carrier, and a first deliquescent; and a second biocidal mixture comprised of a second active biocidal ingredient, a second carrier, and a second deliquescent. The first biocidal mixtures is prepared by mixing and heating the first active biocidal ingredient, the first carrier, and the first deliquescent and then grinding the first biocidal mixture into a plurality of first biocidal particles approximately 1-50 microns in size. The second biocidal mixtures is prepared by mixing and heating the second active biocidal ingredient, the second carrier, and the second deliquescent and then grinding the second biocidal mixture into a plurality of second biocidal particles approximately 1-50 microns in size. Wherein when the first biocidal particles and second biocidal particles are dispersed in the air the first deliquescent and second deliquescent react with ambient humidity to start a chemical reaction within the first and second biocidal particles, wherein the first active biocidal ingredient within the first biocidal particle reacts with the second active biocidal ingredient of the second biocidal particle to create a chemical byproduct which remediates the biological particles.

In a still further aspect of the present invention there is provided a first biocidal mixture comprised of a first active biocidal ingredient, a first carrier, and a first deliquescent; and a second biocidal mixture comprised of a second active biocidal ingredient, a second carrier, and a second deliquescent. The first biocidal mixtures is prepared by mixing and heating the first active biocidal ingredient, the first carrier, and the first deliquescent and then grinding the first biocidal mixture into a plurality of first biocidal particles approximately 1-50 microns in size. The second biocidal mixtures is prepared by mixing and heating the second active biocidal ingredient, the second carrier, and the second deliquescent and then grinding the second biocidal mixture into a plurality of second biocidal particles approximately 1-50 microns in size. Wherein when the first biocidal particles and second biocidal particles are dispersed in the air the first deliquescent and second deliquescent react with ambient humidity to start a chemical reaction wherein the first biocidal particles create a first chemical byproduct for remediation of a first biological particle and the second biocidal particle creates a second chemical byproduct for remediation of a second biological particle.

In another aspect of the present invention the carrier of the biocidal mixture is diatomaceous Earth, the active biocidal ingredient is sodium chlorite, the deliquescent is sodium chloride, and the biocidal mixture also contains calcium chloride; and wherein the carrier of the second biocidal mixture is diatomaceous Earth, the active biocidal ingredient is citric acid, the deliquescent is calcium chloride, and the second biocidal mixture also contains sodium chloride.

In another aspect of the present invention, the biocidal mixture creates a chemical byproduct of chlorine dioxide.

In another aspect of the present invention, the biocidal mixture creates a chemical byproduct of hydrogen peroxide.

In another aspect of the present invention, the biocidal mixture is comprised of a carrier of diatomaceous Earth, an active biological ingredient of sodium percarbonate, and a deliquescent of calcium chloride. The biocidal mixture does not have to by anhydrous.

In another aspect of the present invention, the biocidal mixture is comprised of carrier of diatomaceous Earth, active biological ingredient which is botanical, and a deliquescent of sodium chloride. In a still further aspect, the active biological ingredient is Cinnamon leaf oil or Lemongrass oil.

In another aspect of the present invention there is provided a method for remediating a plurality of air-dispersed biological particles comprising the steps of: preparing a biocidal mixture containing at least one carrier, one active biological ingredient and one deliquescent; grinding said biocidal mixture into a plurality of biocidal particles approximately 1-50 microns in size; electrostatically charging said biocidal particles upon dispersion so that said biocidal particles attract said biological particles; and dispersing said biocidal particles into the air infected with said biological particles; wherein said deliquescent reacts with ambient humidity to create a chemical reaction within said biocidal particle creating a chemical byproduct which remediates said biological particles.

In a still further aspect of the present invention there is provided a method further for remediation of a plurality of air-dispersed biological particles comprising the steps of: preparing a first biocidal mixture containing a first carrier, a first active biocidal ingredient and a first deliquescent; grinding said first biocidal mixture into a plurality of first biocidal particles approximately 1-50 microns in size; preparing a second biocidal mixture containing a second carrier, a second active biocidal ingredient and a second deliquescent; grinding said second biocidal mixture into a plurality of second biocidal particles approximately 1-50 microns in size; charging said first biocidal particles and said second biocidal particles upon dispersion into the air infected with said biological particles so that the first biocidal particles and the second biocidal particles attract the biological particles; wherein the first deliquescent and second deliquescent react with ambient humidity and said first active biocidal ingredient reacts with said second active biocidal ingredient to create a chemical byproduct which remediates said biological particles.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a depiction of air dispersed biocidal particles attracting and remediating dispersed biological particles.

### DETAILED DESCRIPTION OF THE PREFERRED EMBODIMENT

As deployed for airborne protection, the present invention provides a biocidal particle designed to be an attractant to free floating spores or other biological forms on its own or attached to a carrier. The attraction may be due to electrical charges or affinities, physical shape (i.e.: fostering surface bonding or binding), hydrophilic of other such forces. The biocidal particle can be formulated as a dry mix for ease in storage and transportation but the biocidal mixture does not have to be formulated as a dry mix. Once the biocidal particles are placed in ambient humidity they go through a chemical reaction which causes the release of various byproducts, such as chlorine dioxide or hydrogen peroxide, which have the ability to attack and remediate the biological particles or spores. The present invention can also employ more than one type of biocidal particle which not only reacts with ambient humidity but also reacts with each other to produce desired chemical byproducts, such as chlorine dioxide, for attacking and remediating the biological particles without the hazards of transporting unstable or harmful substances.

The present invention as deployed for surface remediation would include a biocidal particle designed to be spread over surface areas in an aerosol or dry mix embodiment. As the particle is exposed to ambient humidity or surface moisture, the particle (salts, etc.) dissolves effectively spreading biocidal agents over an area several times larger than a solid particle. Complete surface coverage is possible with dispersal of biocidal particles.

In a preferred embodiment the Biocidal particles will be approximately 1-50 microns in size, may be any geometric shape created by crystallization of salts or any shape created by diatoms (i.e. diatomaceous earth) and may be ground to any size between 1-50 microns. The Biocidal particles may also be spray dried to any size. In the preferred embodiment the Biocidal particles will be a dark color as this will allow solar heating to energizing the particles. The particles may be charged positively, negatively, or remain neutral.

Biocidal Particle Composition

The biocidal particle in its primary form is comprised of three component parts: (1) the carrier, (2) the active biocidal ingredients, and (3) the deliquescent. The carrier is typically a microporous inorganic that will increase surface area for chemical “painting.” Essentially, during the process of mixing the components some impregnation will occur into the carrier or microporous particle. The impregnation will achieve a coating of 1 the walls of the pores thereby increasing the surface area of the particle. The treated surfaces then allow or provide more points of contact and slow the release time (dwell time) of the biocidal chemical gas. The carrier may also provide physical bonding or binding points on its surface. The active biocidal ingredients are the chemical biocides. The deliquescent compounds render the particle hydrophilic and will facilitate dissolution on surface areas. Provided below are partial lists of various carriers, active biocidal ingredients and deliquescent substances which may be used. The lists are not intended to be limiting and are merely intended as an exemplary list to identify the types and kinds of carriers, active biocidal ingredients, and deliquescent which may be used.

Partial List of Carriers

| | | |
|---|---|---|
| Diatomaceous Earth | Kaolin | Zeolites |
| Pearlites | Bentonite | Charcoal |
| Super Absorbents | Chabazite | Table Salt |
| | | (sodium chloride) |
| Calcium Chloride | Dolomite | Vermiculite |

Partial List of Active Biocidal Ingredients

| | | |
|---|---|---|
| Cedar Oil | Cinnamon Oil | Citronella Oil |
| Eugenol | Geranium Oil | Lemongrass Oil |
| Mint Oil | Potassium Sorbate | Rosemary Oil |
| Thyme Oil | Citrol | Limonene |
| Acetic Acid | Ascorbic Acid | Citric Acid |
| Tartaric Acid | Malic Acid | Glyolic Acid |
| Sorbic Acid | Glutaric Acid | Succinic Acid |
| Pentenoric Acid | Butyric Acid | Sulfuric Acid |
| Phosphoric Acid | Propionic Acid | Benzoic Acid |
| Lactic Acid | Sodium Percarbonate | Sodium Chlorite |

Partial List of Deliquescent

| | | |
|---|---|---|
| Calcium Chloride | Sodium Chloride | Lithium Chloride |
| Magnesium Nitrate | Alumina | Magnesium Chloride |

| | |
|---|---|
| Calcium Chloride | Silica Gel and |
| | mixtures there of |

In a preferred embodiment the application will employ the use of two active biocidal ingredients which work or react in combination to form the desired chemical byproduct for remediation of the biological particles or spores. In the preferred embodiment each active biocidal ingredient is prepared and stored separately. The first active biocidal mixture will preferably be comprised of one (1) part carrier of diatomaceous Earth, one (1) part active biocidal ingredient of sodium chlorite, one (1) part deliquescent of sodium chloride, and one (1) part calcium chloride and an additional ingredient to create the desired by product. The sodium chloride could be modified by adding a colorant. The second active biocidal mixture will be comprised of one (1) part carrier of diatomaceous Earth, one (1) part active biocidal ingredient of citric acid, one (1) part deliquescent of calcium chloride, and one (1) part sodium chloride to help create the desired byproduct. The proper amount of ingredients in the mixtures of the preferred embodiment are determined by weight such that its one part carrier, one part active, and one part deliquescent by weight. When the finely ground particles of the first active biocidal mixture are released with the finely ground particles of the second active biocidal mixture into air at ambient humidity the first and second active biocidal ingredients will react with each other to create chlorine dioxide. Chlorine Dioxide is a known substance which can remediate biological agents.

In another embodiment, the mixture would be comprised of one (1) part carrier such as diatomaceous Earth, one (1) part active biocidal ingredient of sodium percarbonate, and one (1) part deliquescent of calcium chloride. This mixture will create hydrogen peroxide when exposed to ambient humidity.

A still further exemplary embodiment comprises a mixture with one (1) part carrier, one (1) part botanical active biocidal ingredient (such as cinnamon leaf or lemongrass), and one (1) part deliquescent of calcium chloride (modified with colorant). The mixture of this embodiment is biocidal immediately due to the botanical active biocidal ingredient.

As previously mentioned, the present invention can employ the use of more than one biocidal particle or active biocidal ingredient in combination with another active biocidal ingredient. For example, the use of Sodium Chlorite, Sodium Chlorate, Ferric Sulfate, or Ferric Chloride in combination with any acid creates chlorine dioxide. In addition, the use of Sodium Perborate or Sodium Percarbonate in combination with water creates hydrogen peroxide. Hydrogen peroxide in combination with citric acid or acetic acid creates paracitric and paracetic acids. Therefore, the biocidal particles can be chosen to work in combination with other separate biocidal particles or the biocidal particles could have two active biocidal ingredients within the same particle which will react with one another when exposed to ambient humidity. The reaction to ambient humidity will provide the desired results or byproducts which attack and remediate the biological spores and/or particles.

Therefore, the biocidal particles can be selected and prepared as needed to combat the biological agent which needs remediation. If only one biological agent dispersed in the air the present invention may utilize only particles with one active biocidal ingredient. Alternatively, if the ideal chemical byproduct for remediation of a given biological agent is an unstable compound, such as chlorine dioxide, the present invention can be configured as two separate biocidal mixtures each with its own active biocidal ingredient. When the particles from the two separate biocidal mixtures are released into biological agent infested air the two active biocidal ingredients of the two separate biocidal mixture particles react with one another to create the desired remediating byproduct. Additionally, if more than one type of biological agent is present or is feared to have been released there may be a preferred active biocidal ingredient for each different biological agent. Therefore, separate mixtures each containing the preferred active biocidal ingredient could be dispersed for remediation. The different biocidal particles could be dispersed as a grouped mixture, separate but simultaneously, or separate and in a staggered deployment.

To create the compounds or dry biocidal particles mixture the components of each formulation are preferably physically mixed in dry form. The combined mixture is then heated to approximately 100° C. The mixture is then ground into preferred particle sizes, typically 1-50 microns.

Three separate tests were conducted to test the viability of the present invention as a means for attracting and decontaminating biological agents. The tests include a surface zone of inhibition test, a surface efficacy of separate particles test, and an enclosed air test. The results of those tests are summarized below.

Test Results

First, a test inoculant or imitation biological agent mixture was prepared which provides similar qualities to Anthrax. The test inoculant mixture included tryptic soy agar, dry kaolin, subtiles, and distilled water which was prepared and incubated at 25° C. for 7 days. Bacillus subtilis (B. subtilis) are the most commonly used simulant for Anthrax. The mixture was then heat shocked at 85+/−2° C. for 10 minutes to destroy vegetative cells. The dried test inoculant mixture was then ground into particle sizes of 1-20 microns.

Plate Preparation

The test inoculant mixture was then sprinkled approximately one (1) particle per every one (1) centimeter onto a prepared plate of tryptic soy agar.

In the surface zone of inhibition test, each test plate was then treated with 0.10 ml biocidal particles placed in a pile in the center of the prepared plate or agar dish to determine zone of inhibition (ZOI). The various active biocidal ingredients tested included Cinnamon Leaf, a chlorine dioxide emitting compound, a hydrogen peroxide emitting compound, and Lemongrass. In addition, two control samples were tested which consisted of Kaolin. As seen by the test results provided below, most of the active biocidal ingredients tested produced no growth and only the Lemongrass produced a zone of inhibition of 2.7 cm.

Test Results—Zone of Inhibition

| | Active biocidal | Average |
|---|---|---|
| Sample | ingredient | length ZOI |
| #1 | Cinnamon Leaf | 4 cm (no growth) |
| #2 | CLO₂ emitting | 4 cm (no growth) |
| | compound | |
| #3 | Hydrogen Peroxide | 4 cm (no growth) |
| | emitting cmpd. | |
| #4 | Lemongrass | 2.7 cm's |
| Control sample #1 | Kaolin only | 0* |
| Control sample #2 | Kaolin only | 0* |

*bacterial lawn over entire plate

In the surface efficacy test, each test plate was treated with 0.10 ml biocidal particles which were sprinkled randomly across the surface of the prepared plate or agar dish to determine efficacy as separate particles. Once again, the active biocidal ingredients tested included Cinnamon Leaf, a chlorine dioxide emitting compound, a hydrogen peroxide emitting compound, and Lemongrass. Kaolin was used for the two control samples. As evident by the test results provided below there was no growth on the plates for any of the active biocidal ingredients tested. In contrast the two Kaolin tests resulted in a full bacterial growth or lawn.

Test Results—Efficacy

| | Active biocidal | Average |
|---|---|---|
| Sample | ingredient | length ZOI |
| Sample #1 | Cinnamon Leaf | <1 (no growth) |
| Sample #2 | CLO₂emitting | <1 (no growth) |
| | compound | |
| Sample #3 | Hydrogen Peroxide | <1 (no growth) |
| | emit. cmpd. | |
| Sample #4 | Lemongrass | <1 (no growth) |
| Control sample #1 | .10 ml Kaolin | full bacterial lawn |
| Control sample #2 | .10 ml Kaolin | full bacterial lawn |

The final test conducted was a random air interaction simulation test to demonstrate that when the simulant material mixes with the active particle in the air the resultant material is rendered harmless. First, dried test inoculant particles sized about 0.10 ml were placed into a 1 cubic foot empty container. Next, active biocidal particles sized about 0.10 ml were placed into the same 1 cubic foot container with the test inoculant. The container was shaken for 1 minute to allow the biocidal and test inoculant particles to interact. Half of the precipitated material in the container was then sprinkled onto a prepared agar plate and incubated for 48 hours with the following results.

Test Results—Random Interaction

| | Active biocidal | Average |
|---|---|---|
| Sample | ingredient | length ZOI |
| Sample #1 | Cinnamon Leaf | no growth |
| Sample #2 | CLO₂ emitting | no growth |
| | compounds | |
| Sample #3 | Hydrogen Peroxide | no growth |
| | emit. cmpds. | |
| Sample #4 | Lemongrass | no growth |
| Sample #5 | Control | 3.7 × 10 to |
| | | the 7^{th} CFU/ml |

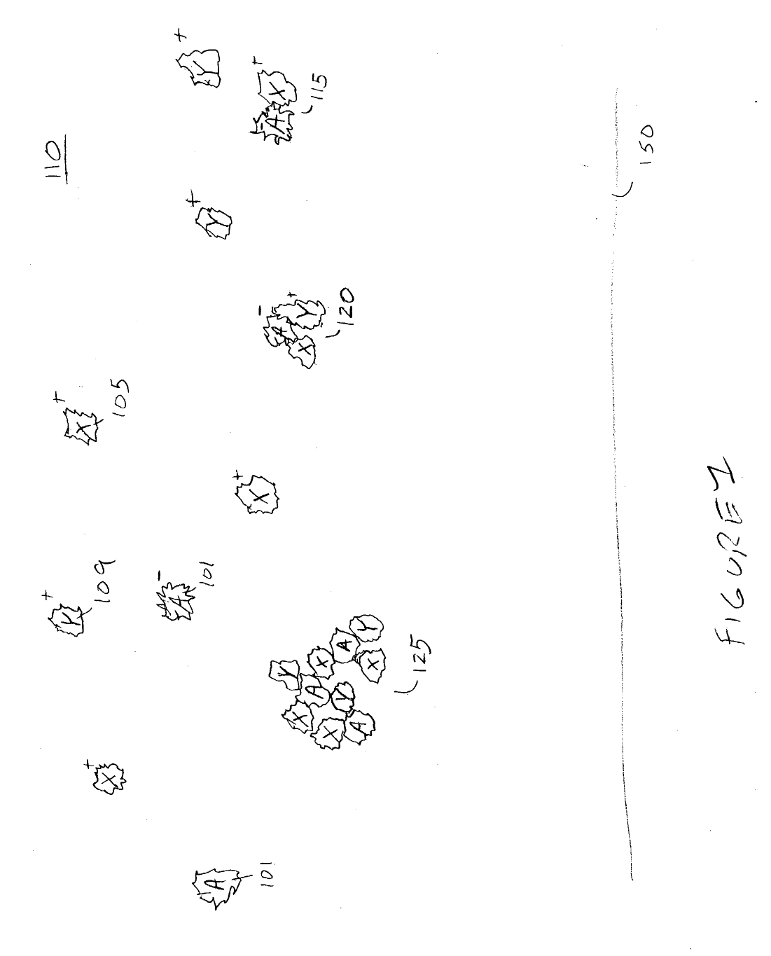

To illustrate the usefulness of the present invention a complete exemplary application utilizing the preferred embodiment of the present invention against the biological agent Anthrax will now be discussed in conjunction with FIG. 1.

Anthrax is a disease caused by bacteria which is relatively harmless while in the ground or on the surface. Once the anthrax spores come into contact with the right environment they begin to germinate. Anthrax may enter the human body through inhalation; cutaneous infection, such as entry into an open cur or wound; or through ingestion of contaminated food. However, the more likely threat of exposure to Anthrax is from manufactured Anthrax developed as a weapon of mass destruction which is then dispersed in the air over a geographical area. “Weaponized Anthrax” has been prepared in a manner that generates ultra fine particles which can remain suspended in the air for long periods of time—thus maximizing their ability to disperse and create inhalation exposure.

Inhalation of Anthrax into the lungs requires very small spores sized approximately 1 to 5 microns and as many as 2500 spores may have to be inhaled to cause an infection. Therefore, the expected dispersion of Anthrax as a weapon would be the deployment of billions of Anthrax spores 101 or particles sized in the 1-50 micron range. The Anthrax particles 101 will likely be positively or negatively charged and will be free floating in the air 110. To make the area or location safe again, the Anthrax spores 101 need to be removed from the air 110 and remediated.

In the preferred embodiment, two active biocidal ingredient compounds are used to remediate the Anthrax. The first active biocidal ingredient compound is prepared by mixing 1 part of diatomaceous Earth as a carrier, 1 part sodium chlorite, 1 part sodium chloride which has been modified with a colorant, and 1 part calcium chloride. The first active biocidal ingredient mixture is heated to 100° C. and then ground into minute particle sizes approximately 1-50 microns. The second active biocidal ingredient compound is made my mixing one part diatomaceous Earth as a carrier, 1 part sodium chloride which has been modified with a colorant, 1 part citric acid, and 1 part calcium chloride. The second active biocidal ingredient mixture is heated to 100° C. and then ground into minute particle sizes approximately 1-50 microns. The compounds are still separate and in dry form. Each compound may be stored in dry sacks or bags such as a 2000 lb super sack or a 50 lb water resistant lined bag. The bags would be sealed and stored in a dry location.

Once the Anthrax is released into the environment over a given area the two active biocidal ingredient compounds of the preferred embodiment are transported and deployed into the Anthrax cloud or air 110. Deployment may utilize an aerosol generator and/or employ the use of a crop duster, other aircraft, or any other means of transportation. Typically, the dry mix ingredients of both active biocidal ingredient compounds would be combined at the Anthrax cloud 110 and dispersed using an aerosol generator, sand blaster, or similar type device. The biocidal particles 105, 109 begin to react immediately when they are premixed prior to or at the time of release. The biocidal particles 105, 109 will also mix in the air with each other by hydrophilic action. Further, when the mixed biocidal particles 105, 109 are released into the Anthrax infected air 110 the biocidal particles 105, 109 are charged positively or negatively to be electrically opposite of the charge of the free floating Anthrax spores 101. The opposite positive or negative charge attracts the Anthrax spores 101 and the shape of the diatomaceous Earth carrier, which contains many points and binder sites, causes the Anthrax spores 101 to become attached thereto. The billions of Anthrax spores 101 and biocidal particles 105, 109 begin to create partial groupings 115, complete groupings 120, and large groupings 125.

In addition, at the same time the Anthrax spores 101 and biocidal particles 105, 109 are creating grouped particles 115, 120, 125 the biocidal particles 105, 109 are reacting to the humidity in the air as a result of the hydrophilic qualities of the particles. As the hydrophilic reaction takes place the sodium chlorite of the first active biocidal ingredient compound 105 reacts with the citric acid of the second active biocidal ingredient compound 109 to form chlorine dioxide. The general chemical equation for this process is provided below:

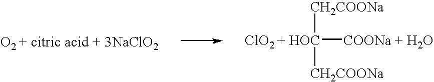

The resultant chlorine dioxide then attacks the Anthrax spores 101 by breaking the outer shell of the Anthrax spore 101 and breaking the spore 101 into nontoxic pieces. Further, as the grouped Anthrax spores and active particles 115, 120, 125 grow in size the Anthrax spores 101 are not only remediated but also begin to precipitate to the surface 150. Once on the surface 150 the chlorine dioxide will act as an inhibitor to any Anthrax growth. Therefore, the present invention enables the air 110 and surface 150 of an infected region to be remediated of air dispersed biological agents 101 such as Anthrax.

The present invention provides an easily transportable and deployable solution for attracting and remediating biological agents such as Anthrax. The present invention is particularly useful in combating large scale dissemination of air dispersed biological agents over large areas. In addition to the preferred embodiment discussed above, other exemplary embodiments include a dry mix compound which creates hydrogen peroxide when exposed to ambient humidity, or the use of a botanical extracts such as Cinnamon Leaf or Lemongrass. Cinnamon Leaf and Lemongrass are biocidal immediately and do not need ambient humidity to begin a biological particle breakdown process.

A still further dissemination method would be the incorporation of a dual process whereby biocidal particles of the present invention are dispersed in the air, such as through use of an aerosol configuration, to attach to the Anthrax spores and cause (1) an automatic chemical process to remediate the spores, and (2) cause the attached particles to become large enough that they precipitate to the surface. To ensure that all of the spores are remediated the surface could also be treated which biocidal particle compounds of the present invention. Applying the compounds to the surface could still be conducted through airborne dissemination where the particle size is large enough that the biocidal particle is likely to settle or fall to the surface.

The various biocidal chemicals such as chlorine dioxide, hydrogen peroxide, and ozone are all oxidizing chemicals that break the molecular bonds of the biological spores, cut through the protein shells and kill the organism.

The various biocidal particles have different activation times and efficacy periods. For example, the chlorine dioxide particles formed from mixing the first and second mixture of the preferred embodiment is active immediately when the first and second mixtures are combined. The mixing might occur in a hopper, pre-feed attachment or in the spreader of an aircraft for large scale air deployment. Hydrogen peroxide particles are active immediately when exposed to ambient humidity. Both chlorine dioxide and hydrogen peroxide will have an efficacy period of up to four (4) days from the time of deployment and the various oil extract particles have an efficacy period of up to 3 months from the date of manufacture. The efficacy period of all the biocidal actives is dependent upon the ambient humidity, levels of moisture on the ground, loading of biological agent and the weather.

In addition to methods of deployment and uses discussed above the present invention could also be used to create protective clothing, filters, and paper by layering the impregnated biocidal particles into paper or fabric. The impregnated fabric would be useful for those treating or working with biological agents, the paper would be useful for testing the presence of biological agents, and the filters would be useful for minimizing the dispersion of agents in contained buildings with forced air systems. Once again, the filters could be formed using paper, fibers, or fabrics impregnated with the biocidal particles. The oil extract actives may be most beneficial in filters as they have a 3 month efficacy period. The particles within the filters could be charged to attract agents and multiple filters each with an opposite charge or active could be placed in the same flow. Another use would of the biocidal particles of the present invention would be to impregnate the biocidal particles in a filter for a gas mask of the like for use by those treating or working with the biological agents.

While the invention has been described in detail and with reference to specific embodiments thereof, it will be apparent to those skilled in the art that various changes and modifications can be made therein without departing from the spirit and scope thereof. Thus, it is intended that the present invention cover the modifications and variations of this invention provided they come within the scope of the appended claims and their equivalents.

What is claimed is:

1. A biocidal mixture for remediating a plurality of biological particles comprising:
at least one carrier, at least one active biocidal ingredient and at least one deliquescent; wherein said at least one carrier, said at least one active biocidal ingredient and said at least one deliquescent are mixed, heated and ground into a plurality of biocidal particles approximately 1-50 microns in size;
wherein when said plurality of biocidal particles are dispersed in the air said deliquescent reacts with ambient humidity to start a chemical reaction within said biocidal particle creating a chemical byproduct which remediates said biological particles.

2. The biocidal mixture of claim 1, further comprising:
a second biocidal mixture containing at least one second carrier, at least one second active biocidal ingredient and at least one second deliquescent; wherein said at least one second carrier, said at least one second active biocidal ingredient and said at least one second deliquescent are mixed, heated and ground into a plurality of second biocidal particles approximately 1-50 microns in size; and
wherein when said plurality of second biocidal particles are dispersed in the air said second deliquescent reacts with ambient humidity to start a chemical reaction within said second biocidal particle; and
said second biocidal particle reacts with said biocidal particle to create said chemical byproduct which remediates said biological particles.

3. The mixture of claim 2, wherein said at least one carrier is diatomaceous earth, said at least one active biocidal ingredient is sodium chlorite, and said at least one deliquescent is sodium chloride; and
wherein said at least one second carrier is diatomaceous Earth, said at least one second active biocidal ingredient is citric acid, and said at least one deliquescent is calcium chloride.

4. The biocidal mixture of claim 2, wherein said chemical byproduct is chlorine dioxide.

5. The biocidal mixture of claim 1, wherein said carrier of said biocidal mixture is diatomaceous Earth, said active is sodium percarbonate, and said deliquescent is calcium chloride.

6. The biocidal mixture of claim 1, wherein said carrier of said biocidal mixture is diatomaceous Earth, said active biocidal ingredient is botanical, and said deliquescent is calcium chloride.

7. The biocidal mixture of claim 6, wherein said active biocidal ingredient is Cinnamon leaf.

8. The biocidal mixture of claim 6, wherein said active biocidal ingredient is Lemongrass.

9. The biocidal mixture of claim 1, wherein said chemical byproduct is chlorine dioxide.

10. The biocidal mixture of claim 1, wherein said chemical byproduct is hydrogen peroxide.

11. A method for remediating a plurality of air dispersed biological particles comprising the steps of:
preparing a biocidal mixture containing at least one carrier, at least one active biocidal ingredient and at least one deliquescent;
grinding said biocidal mixture into a plurality of biocidal particles approximately 1-50 microns in size;
charging said biocidal particles upon dispersion so that said biocidal particles attract said biological particles; and
dispersing said biocidal particles into air infected with said biological particles;
wherein said at least one deliquescent reacts with ambient humidity to create a chemical reaction within said biocidal particle creating a chemical byproduct which remediates said biological particles.

12. The method of claim 11, further comprising the step of:
preparing a second biocidal mixture containing at least one second carrier, at least one second active biocidal ingredient and at least one second deliquescent;
grinding said second biocidal mixture into a plurality of second biocidal particles approximately 1-50 microns in size;
charging said biocidal particles and said second biocidal particles upon dispersion so that said biocidal particles and said second biocidal particles attract said biological particles; and
dispersing said biocidal particles and said second biocidal particles into the air infected with said biological particles; wherein said biocidal particles react with said second biocidal particles to create a chemical byproduct which remediates said biological particles.
